(19) United States
(12) Patent Application Publication
(10) Pub. No.: US 2026/0056159 A1
(43) Pub. Date: Feb. 26, 2026

(54) ENZYMATIC REACTION DEVICE AND ENZYMATIC REACTION METHOD

(71) Applicant: Panasonic Intellectual Property Management Co., Ltd., Osaka (JP)

(72) Inventors: FUMIYA WAYAMA, Osaka (JP); NORIYUKI HATSUGAI, Kyoto (JP); YASUAKI OKUMURA, Kyoto (JP); KIYOTAKA TSUJI, Osaka (JP); KUNIHIRO UKAI, Nara (JP)

(21) Appl. No.: 19/378,413

(22) Filed: Nov. 4, 2025

Related U.S. Application Data
(63) Continuation of application No. PCT/JP2024/015695, filed on Apr. 22, 2024.

(30) Foreign Application Priority Data
May 16, 2023 (JP) 2023-080659

(51) Int. Cl. G01N 27/327; C12Q 1/00; C12Q 1/26; G01N 33/02

(52) U.S. Cl. CPC G01N 27/3273; C12Q 1/005; C12Q 1/26; G01N 27/3276; G01N 33/02

(57) ABSTRACT

An enzymatic reaction device includes first and second electrodes and a voltage applicator that applies a voltage to the first and second electrodes. The first and second electrodes each include at least one of an enzyme or a coenzyme that causes a reaction of a target molecule in a sample, and an electrode body including at least one of the enzyme or coenzyme immobilized on its surface. The voltage application by the voltage applicator is performed in such a manner that a first period during which a voltage is applied to the first electrode so that the first electrode functions as a working electrode that causes a reaction of the target molecule, and a second period during which a voltage is applied to the second electrode so that the second electrode functions as a working electrode that causes a reaction of the target molecule, are alternately repeated.

### BACKGROUND

### 1. Technical Field

The present disclosure relates to an enzymatic reaction device and a method for performing an enzymatic reaction.

### 2. Description of the Related Art

There is known a technique for performing a reaction of a target molecule in a sample using an electrode containing at least one of an enzyme or a coenzyme that causes a reaction of a target molecule. For example, International Publication No. 2021/261509 (Patent Document 1) discloses a technique for efficiently reducing disulfide bonds in a protein by using a working electrode containing an enzyme that reduces disulfide bonds in a protein and applying a voltage to the working electrode.

### SUMMARY

In an enzymatic reaction using an enzyme or coenzyme immobilized on an electrode, it is necessary to improve the reaction efficiency. For example, in a reaction system that contains a large number of molecules other than the target molecule, such as food, the enzymatic reaction may be less likely to proceed, and there is much room for improvement in the efficiency of the enzymatic reaction.

One non-limiting and exemplary embodiment provides an enzymatic reaction device and a method for performing an enzymatic reaction, in which the device and the method can cause a reaction of a target molecule with high efficiency.

In one general aspect, the techniques disclosed here feature an enzymatic reaction device including a first electrode and a second electrode each including at least one of an enzyme or a coenzyme that causes a reaction of a target molecule in a sample, and an electrode body including at least one of the enzyme or the coenzyme immobilized on a surface of the electrode body, and a voltage applicator that applies voltages to the first electrode and the second electrode, in which the voltage applicator applies a voltage to the first electrode and the second electrode in such a manner that a first voltage application period during which a voltage is applied to the first electrode so that the first electrode functions as a working electrode that causes a reaction of the target molecule, and a second voltage application period during which a voltage is applied to the second electrode so that the second electrode functions as a working electrode that causes a reaction of the target molecule, are alternately repeated.

According to an embodiment of the present disclosure, a target molecule can be reacted with high efficiency.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 illustrates an example of a reactor for performing an enzymatic reaction;

FIG. 2 illustrates an example of a voltage application pattern to a working electrode;

FIG. 3 illustrates another example of a voltage application pattern to the working electrode;

FIG. 4 illustrates yet another example of a voltage application pattern to the working electrode;

FIG. 5 illustrates a configuration of an enzymatic reaction device according to an embodiment;

FIG. 6A is a sectional view illustrating a configuration of a first electrode according to an embodiment;

FIG. 6B is a sectional view illustrating a configuration of a second electrode according to the embodiment;

FIG. 7 is a diagram for explaining an enzyme layer immobilized on an electrode body according to an embodiment;

FIG. 8 illustrates an example of a voltage application pattern to the first electrode and the second electrode in the enzymatic reaction device according to an embodiment;

FIG. 9 illustrates a configuration of an enzymatic reaction device according to a variation of the embodiment;

FIG. 10 is a graph illustrating the reduction rates of disulfide bonds in β-lactoglobulin in an example and a comparative example; and

FIG. 11 illustrates changes in the pH of samples in the example and the comparative example.

### DETAILED DESCRIPTIONS

### Background to an Aspect of the Present Disclosure

Before specific description of an embodiment of the present disclosure, background to an aspect of the present disclosure will be described below. The inventors have found that when a reaction of a target molecule is performed using an electrode including an enzyme or a coenzyme immobilized on its surface, the following problems arise.

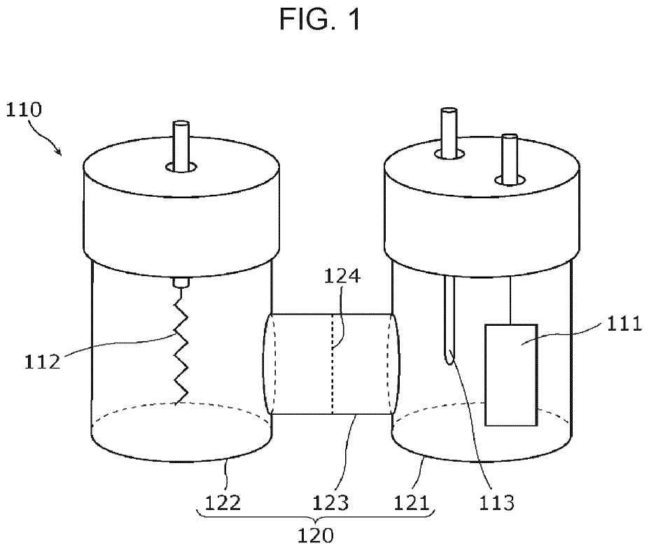

FIG. 1 illustrates an example of a reactor for performing an enzymatic reaction. As illustrated in FIG. 1, a reactor 110 includes a working electrode 111, a counter electrode 112, a reference electrode 113, a reaction vessel 120, and a separator 124. The reaction vessel 120 includes a first container 121, a second container 122, and a connection portion 123. The working electrode 111 and the reference electrode 113 are disposed inside the first container 121. The counter electrode 112 is disposed inside the second container 122. A sample containing a target molecule is placed in the first container 121, and an enzymatic reaction takes place. The first container 121 and the second container 122 are connected through the connection portion 123. The separator 124 disposed at the connection portion 123 inhibits some components from moving between the first container 121 and the second container 122.

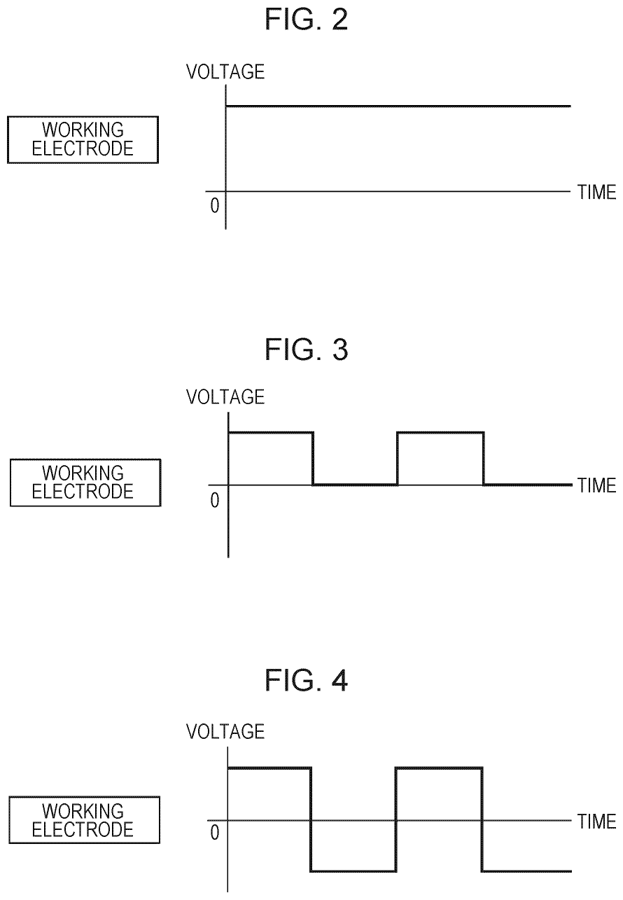

An oxidoreductase that oxidizes or reduces a target molecule is immobilized on the working electrode 111. In the reactor 110, a voltage is applied between the working electrode 111 and the counter electrode 112 to control the potential difference between the working electrode 111 and the reference electrode 113 to a predetermined value. This causes electron transfer between the working electrode 111 and the oxidoreductase, repeatedly activating the oxidoreductase that has oxidized or reduced the target molecule. FIG. 2 illustrates an example of a voltage application pattern to the working electrode 111. For example, as illustrated in FIG. 2, a constant voltage is continuously applied to the working electrode 111. This is also the voltage application pattern disclosed in Patent Document 1 above.

When only the target molecule is present in a sample, the enzymatic reaction proceeds relatively efficiently even with the voltage application pattern illustrated in FIG. 2. However, in a reaction system in which a large number of molecules other than the target molecule are present in a sample, for example, in the case where the sample is food, these molecules are electrically attracted to and adsorbed on the working electrode 111. For example, molecules other than the target molecule contained in food, such as proteins, amino acids, sugars, and lipids, are adsorbed on the working electrode 111. As a result, the target molecule is less likely to approach the working electrode 111. This significantly slows down the progress of the enzymatic reaction, decreasing the reaction efficiency. For example, after a predetermined amount of time has elapsed since the start of the reaction, the enzymatic reaction hardly proceeds at all.

FIGS. 3 and 4 illustrate other examples of voltage application patterns to the working electrode 111. For example, as illustrated in FIGS. 3 and 4, in order to inhibit the adsorption of molecules other than the target molecule on the working electrode 111, it is conceivable to provide a refresh period in which a voltage of 0 V or a voltage with a reversed polarity to that during the progress of the reaction is applied to the working electrode 111. During the refresh period, no voltage is applied to the working electrode 111 that would attract molecules other than the target molecule, so that the molecules other than the target molecule move away from the working electrode 111, allowing the target molecule to approach the working electrode 111 again. However, during such a refresh period, the enzymatic reaction of the desired target molecule stops, requiring a long processing time, and the reaction efficiency cannot be sufficiently increased.

The present disclosure has been accomplished in consideration of such problems, and provides an enzymatic reaction device and a method for performing an enzymatic reaction, in which the device and method can cause a reaction of a target molecule with high efficiency even in a reaction system in which molecules other than the target molecule are present.

### Overview of the Present Disclosure

As an overview of an aspect of the present disclosure, examples of an enzymatic reaction device and a method for performing an enzymatic reaction according to an embodiment of the present disclosure are described below.

An enzymatic reaction device according to a first aspect of the present disclosure includes a first electrode and a second electrode each including at least one of an enzyme or a coenzyme that causes a reaction of a target molecule in a sample, and an electrode body including at least one of the enzyme or the coenzyme immobilized on the surface of the electrode body, and a voltage applicator that applies a voltage to the first electrode and the second electrode. The voltage applicator applies a voltage to the first electrode and the second electrode in such a manner that a first voltage application period during which a voltage is applied to the first electrode so that the first electrode functions as a working electrode that causes a reaction of the target molecule, and a second voltage application period during which a voltage is applied to the second electrode so that the second electrode functions as a working electrode that causes the reaction of the target molecule, are alternately repeated.

The repetition of the first and second voltage application periods as described above inhibits the adsorption of molecules other than the target molecule in the sample on the second electrode during the first voltage application period and inhibits the adsorption of molecules other than the target molecule in the sample on the first electrode during the second voltage application period. This can inhibit a decrease in the efficiency of the enzymatic reaction caused by the adsorption of such molecules over time. Furthermore, since one of the first and second electrodes functions as a working electrode during the first and second voltage application periods, sufficient time is ensured for the enzymatic reaction to occur, enabling the target molecule in the sample to react with high efficiency.

For example, an enzymatic reaction device according to a second aspect of the present disclosure is the enzymatic reaction device according to the first aspect, in which an oxidoreductase that oxidizes or reduces the target molecule is immobilized on the surface of the electrode body as the enzyme.

In this case, the target molecule in the sample can be oxidized or reduced with high efficiency.

For example, an enzymatic reaction device according to a third aspect of the present disclosure is the enzymatic reaction device according to the second aspect, in which each of the first electrode and the second electrode includes an electron carrier that transports an electron between the electrode body and the oxidoreductase, a first linker in the form of a chain, and a second linker in the form of a chain, the second linker being longer than the first linker. The electron carrier is immobilized on the surface of the electrode body with the first linker interposed therebetween. The oxidoreductase is immobilized on the surface of the electrode body with the second linker interposed therebetween.

In this case, electrons are transported between the oxidoreductase and the electrode body with the electron carrier. This improves the efficiency of electron transport between the oxidoreductase and the electrode body, compared with the case where the oxidoreductase, which has a complex three-dimensional structure, directly exchanges electrons with the electrode body. In addition, since the oxidoreductase is distant from the electrode body, damage of the oxidoreductase can be inhibited.

For example, an enzymatic reaction device according to a fourth aspect of the present disclosure is the enzymatic reaction device according to any one of the first to third aspects, in which the length of the first voltage application period is identical to the length of the second voltage application period.

In this case, the enzymatic reaction of the target molecule can occur evenly on the first and second electrodes to thereby more efficiently cause a reaction of the target molecule.

A method for performing an enzymatic reaction according to a fifth aspect of the present disclosure using a first electrode and a second electrode each including at least one of an enzyme or a coenzyme that causes a reaction of a target molecule in a sample and an electrode body including at least one of the enzyme or the coenzyme immobilized on the surface of the electrode body includes applying a voltage to the first electrode so that the first electrode functions as a working electrode that causes the reaction of the target molecule, and applying a voltage to the second electrode so that the second electrode functions as the working electrode that causes the reaction of the target molecule, in which the applying the voltage to the first electrode and the applying the voltage to the second electrode are alternately repeated.

According to the method for performing an enzymatic reaction, the same effects as those of the above-described enzymatic reaction device can be provided.

A method for performing an enzymatic reaction according to a sixth aspect of the present disclosure is the method for performing an enzymatic reaction according to the fifth aspect, in which the sample is food.

Thus, even when the sample is food and contains a large number of molecules other than the target molecule, the molecules other than the target molecule are less likely to be adsorbed on the first electrode and the second electrode, so that the target molecule in the sample can be reacted with high efficiency.

It should be noted that these comprehensive or specific aspects may be realized by a system, a method, a device, an integrated circuit, a computer program, or a recording medium such as a computer-readable CD-ROM, and may be realized by any combination of a system, a method, a device, an integrated circuit, a computer program, and a recording medium.

Embodiments will be specifically described below with reference to the drawings.

Note that each of embodiments below describes a general or specific example. The numerical values, shapes, materials, elements, arrangement and connection of the elements, steps, and order of the steps, etc., indicated in the following embodiments are given merely by way of illustration and are not intended to limit the present disclosure. In addition, among the elements described in the following embodiments, ones not described in the independent claims, which define broadest concepts, will be described as optional elements. Each drawing is not necessarily a strict illustration. In the drawings, substantially the same elements are given the same reference numerals, and redundant description thereof may be omitted or simplified.

The terms, such as parallel and vertical, indicating the relationship between elements, the terms, such as rectangles, indicating the shapes of the elements, and the numerical values do not represent only strict meanings but mean inclusion of a substantially equal range, for example, a difference on the order of few percent.

### EMBODIMENTS

An enzymatic reaction device and a method for performing an enzymatic reaction according to an embodiment will be described below.

### Configuration

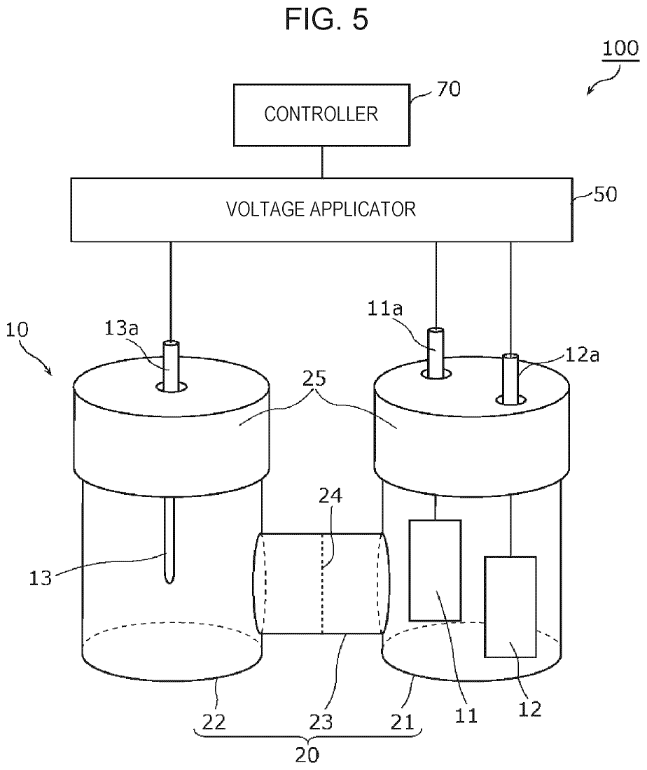

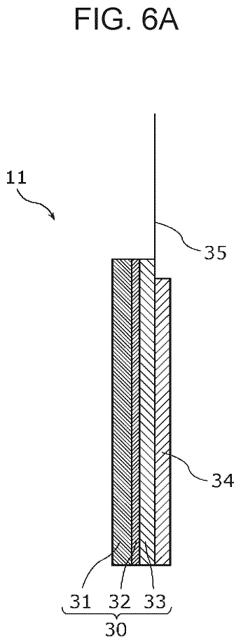

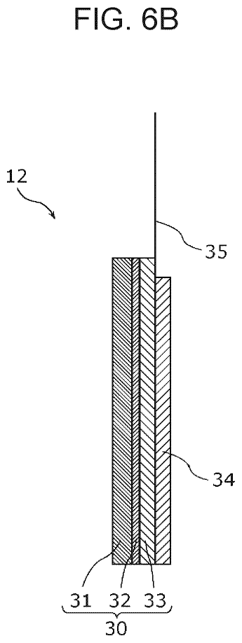

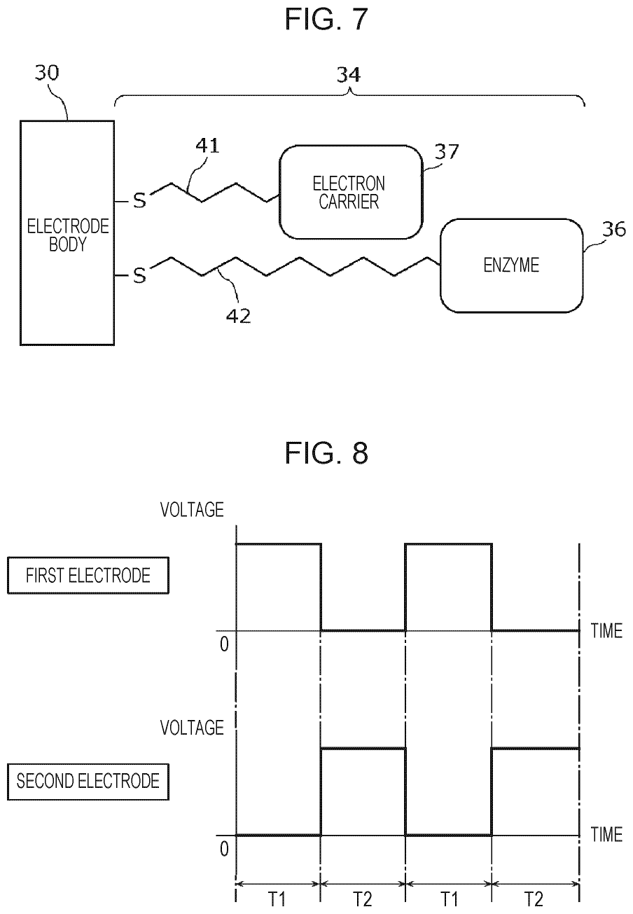

The configuration of the enzymatic reaction device according to the present embodiment will be described with reference to FIGS. 5 to 7. FIG. 5 illustrates a configuration of an enzymatic reaction device according to the present embodiment. FIG. 6A is a sectional view illustrating a configuration of a first electrode according to the present embodiment. FIG. 6B is a sectional view illustrating a configuration of a second electrode according to the present embodiment. FIG. 7 is a diagram for explaining an enzyme layer immobilized on an electrode body according to the present embodiment. In FIG. 5, a first electrode 11, a second electrode 12, and a reference electrode 13 arranged inside a reaction vessel 20 are indicated by solid lines, and portions of the reaction vessel 20 that cannot be seen from the surface are indicated by dashed lines. FIGS. 6A and 6B illustrate sections of the first electrode 11 having a plate-like shape and the second electrode 12 having a plate-like shape cut in the thickness direction. FIG. 7 illustrates a schematic diagram of an enzyme 36 and an electron carrier 37 immobilized on the electrode body 30.

As illustrated in FIG. 5, an enzymatic reaction device 100 according to the present embodiment includes a reactor 10, a voltage applicator 50, and a controller 70. The enzymatic reaction device 100 is a device that causes a reaction of a target molecule in a liquid sample using the reactor 10. The sample is, for example, food. The enzymatic reaction device 100 can, for example, oxidize or reduce the target molecule contained in food to convert the target molecule into another molecule. Examples of food include, but are not limited to, milk, soup, liquid seasoning, soft drinks, alcoholic drinks, and fruit juices. The sample is not limited to food, but may be biological fluid, domestic wastewater, or industrial wastewater.

The reactor 10 includes the first electrode 11, the second electrode 12, the reference electrode 13, terminals 11a, 12a, and 13a, the reaction vessel 20, a separator 24, and container caps 25. In the reactor 10, a voltage is applied to the first electrode 11 and the second electrode 12, causing the reaction of the target molecule in the sample contained in the reaction vessel 20.

The first electrode 11 and the second electrode 12 are enzyme-immobilized electrodes. Each electrode has at least one of an enzyme or a coenzyme that causes the reaction of the target molecule in the sample and that is immobilized on its surface. As illustrated in FIGS. 6A and 6B, the first electrode 11 and the second electrode 12 have, for example, the same structure and each include the electrode body 30, an enzyme layer 34, and a lead 35.

At least one of the enzyme or the coenzyme contained in the enzyme layer 34 is immobilized on the surface of the electrode body 30. The electrode body 30 has, for example, a plate-like shape. The shape of the electrode body 30 is not particularly limited, and may be a shape other than a plate-like shape, for example, a rod-like shape or a mesh-like shape.

In the examples illustrated in FIGS. 6A and 6B, the electrode bodies 30 each include a glass substrate 31, a metal layer 32, and a conductive layer 33. In each of the first electrode 11 and the second electrode 12, the glass substrate 31, the metal layer 32, the conductive layer 33, and the enzyme layer 34 are stacked in that order in the thickness direction. In each of the first electrode 11 and the second electrode 12, at least one of the glass substrate 31 or the metal layer 32 does not necessarily need to be included.

The metal layer 32 is a vapor-deposited film disposed on the surface of the glass substrate 31 using, for example, chromium or titanium. The conductive layer 33 is a conductive substrate disposed on the metal layer 32. The conductive layer 33 is made of, for example, gold, platinum, a carbon material, such as glassy carbon, graphite, or boron-doped diamond, or indium-tin oxide (ITO). The enzyme layer 34 containing at least one of the enzyme or the coenzyme is immobilized on the surface of the conductive layer 33.

The conductive layer 33 is connected to the lead 35. The lead 35 of the first electrode 11 is connected to the terminal 11a illustrated in FIG. 5. The lead 35 of the second electrode 12 is connected to the terminal 12a illustrated in FIG. 5.

As illustrated in FIG. 7, the enzyme layer 34 includes the enzyme 36, the electron carrier 37, a first linker 41, and a second linker 42. FIG. 7 illustrates a part of the enzyme layer 34. In reality, enzymes 36 and electron carriers 37 are immobilized on the electrode body 30. The enzyme layer 34 may further include an enzyme other than the enzyme 36 or a coenzyme.

The enzyme 36 is immobilized on the surface of the electrode body 30 with the second linker 42 interposed therebetween. The enzyme 36 is, for example, an oxidoreductase that oxidizes or reduces a target molecule (that is, a substrate). Examples of the enzyme 36 include ferredoxin-thioredoxin reductase, glucose dehydrogenase, and alcohol dehydrogenase. When the enzyme 36 is ferredoxin-thioredoxin reductase, the target molecule is an allergenic protein having a disulfide bond. Specific examples thereof include B-lactoglobulin, prolamin, and ovalbumin. When the enzyme 36 is glucose dehydrogenase or alcohol dehydrogenase, nicotinamide adenine dinucleotide (NADH) may be contained in the enzyme layer 34 as a coenzyme.

The electron carrier 37 transports electrons between the electrode body 30 and the enzyme 36. The electron carrier 37 is immobilized on the surface of the electrode body 30 with the first linker 41 interposed therebetween. The electron carrier 37 is not particularly limited as long as it is a compound that can mediate electron transfer between the electrode body 30 and the enzyme 36. Examples thereof include methyl viologen, quinone, and indophenol.

The first linker 41 has a chain-like molecular structure with one end chemically bonded to the electrode body 30 and the other end chemically bonded to the electron carrier 37. The first linker 41 includes, for example, an alkyl chain as the main chain. One end of the alkyl chain of the first linker 41 is substituted with, for example, a thiol group. The thiol group forms a metal-sulfur bond with a metal on the surface of the electrode body 30. The other end of the alkyl chain of the first linker 41 is substituted with, for example, a functional group that can bind to the electron carrier 37 and is bonded to the electron carrier 37. In addition, the other end of the alkyl chain of the first linker 41 may be directly substituted with the electron carrier 37.

The second linker 42 has a chain-like molecular structure with one end chemically bonded to the electrode body 30 and the other end chemically bonded to the enzyme 36. The second linker 42 includes, for example, an alkyl chain as the main chain. One end of the alkyl chain of the second linker 42 is substituted with, for example, a thiol group. The thiol group forms a metal-sulfur bond with the metal on the surface of the electrode body 30. The other end of the alkyl chain of the second linker 42 is substituted with, for example, a carboxy group or an amino group. The carboxy group is bonded to the amino group of the enzyme 36 to form an amide bond. The amino group is bonded to the carboxy group of the enzyme 36 to form an amide bond.

The second linker 42 has a chain-like molecular structure that is longer than the first linker 41. The number of carbon atoms in the alkyl chain of the first linker 41 is, for example, greater than or equal to 2 and less than or equal to 5. The number of carbon atoms in the alkyl chain of the second linker 42 is, for example, greater than or equal to 6 and less than or equal to 14.

Since the enzyme layer 34 has the above-described configuration, electrons are transported between the enzyme 36 and the electrode body 30 through the electron carrier 37. Thus, the efficiency of electron transport between the enzyme 36 and the electrode body 30 is improved, compared with the case where the enzyme 36, which has a complex three-dimensional structure, directly transfers electrons to and from the electrode body 30. Furthermore, since the enzyme 36 is distant from the electrode body 30, damage of the enzyme 36 can be inhibited.

In manufacturing each of the first electrode 11 and the second electrode 12 having such a structure, for example, the electrode body 30 is prepared. The electrode body 30 is immersed in a solution containing the first linker 41 bonded to the electron carrier 37 and the second linker 42. This causes the thiol groups of the first linker 41 and the second linker 42 to bond with the conductive layer 33 of the electrode body 30, forming a self-assembled monolayer. The enzyme 36 is allowed to react with the amino group or carboxy group of the second linker 42 to form an amide. This results in an enzyme-immobilized electrode in which the enzyme 36 and the electron carrier 37 are immobilized on the surface of the electrode body 30.

The configurations of the first electrode 11 and the second electrode 12 are not limited to the examples illustrated in FIGS. 6A, 6B, and 7, and are not particularly limited as long as at least one of an enzyme or a coenzyme is immobilized on the surface of each electrode body 30. Examples of the configurations include a configuration in which an enzyme is immobilized on the first electrode 11 and an enzyme is immobilized on the second electrode 12, a configuration in which a coenzyme is immobilized on the first electrode 11 and a coenzyme is immobilized on the second electrode 12, a configuration in which an enzyme is immobilized on the first electrode 11 and a coenzyme is immobilized on the second electrode 12, and a configuration in which a coenzyme is immobilized on the first electrode 11 and an enzyme is immobilized on the second electrode 12.

Referring again to FIG. 5, the reference electrode 13 is an electrode that does not react with components in the sample and maintains a constant potential, and is used to control the potential difference between the reference electrode 13 and the first and second electrodes 11 and 12 at a constant value. The reference electrode 13 is, for example, a silver/silver chloride electrode. In controlling the potential using the reference electrode 13, for example, a mechanism similar to the control mechanism of an electrochemical measurement device, such as a potentiostat, can be used. The reactor 10 does not necessarily need to include the reference electrode 13.

The reaction vessel 20 is a vessel that contains the sample. The reaction vessel 20 includes a first container 21, a second container 22, and a connection portion 23 that connects the first container 21 and the second container 22.

The first electrode 11 and the second electrode 12 are disposed inside the first container 21. The first container 21 contains the sample containing the target molecule. In the first container 21, the reaction of the target molecule occurs with the first electrode 11 and the second electrode 12.

The reference electrode 13 is disposed inside the second container 22. The second container 22 contains, for example, a standard solution. The standard solution is, for example, physiological saline. The standard solution may have a pH buffering capacity. The reference electrode 13 may be disposed inside the first container 21.

In the example illustrated in FIG. 5, the shape of each of the first container 21 and the second container 22 is cylindrical, but is not particularly limited thereto, and may be a shape other than cylindrical, such as a rectangular parallelepiped, an elliptical cylinder, a polygonal cylinder, or a sphere.

The connection portion 23 is a tubular member that connects the inside of the first container 21 and the inside of the second container 22. The separator 24 is disposed in the connection portion 23. The separator 24 inhibits some components of the sample contained in the first container 21 and the standard solution contained in the second container 22 from moving to each other.

The separator 24 does not transmit, for example, the target molecule and a reaction product of the target molecule in the sample, but it transmits some ions, such as protons. The separator 24 is, for example, an ion exchange membrane having ionic conductivity. A specific example of the separator 24 is a membrane made of a polymer having a perfluorinated side chain containing a sulfonic acid group, such as Nafion (registered trademark). The separator 24 may be made of a material other than an ion exchange membrane, such as porous glass or porous silicon.

The container caps 25 are provided on the respective opening portions of the first container 21 and the second container 22, and cover the respective opening portions of the first container 21 and the second container 22. The container caps 25 have through-holes through which the terminals 11a, 12a, and 13a are inserted.

Although not illustrated in the figure, the enzymatic reaction device 100 may be provided with a stirring mechanism that stirs each of the sample contained in the first container 21 and the standard solution contained in the second container 22.

The voltage applicator 50 is a voltage application circuit that applies a voltage to the first electrode 11 and the second electrode 12. The voltage applicator 50 is electrically connected to the first electrode 11 with the terminal 11a interposed therebetween, electrically connected to the second electrode 12 with the terminal 12a interposed therebetween, and electrically connected to the reference electrode 13 with the terminal 13a interposed therebetween. The voltage applicator 50 applies a voltage to the first electrode 11 and the second electrode 12 under the control of the controller 70. As will be explained in detail below, the voltage applied from the voltage applicator 50 to the first electrode 11 and the second electrode 12 can be switched between different voltages during the first voltage application period and the second voltage application period, thereby causing the target molecule to react with high efficiency.

There are no particular limitations on the voltage applicator 50 as long as it is a circuit that can change the voltage applied to the first electrode 11 and the second electrode 12 in a desired pattern. For example, the voltage applicator 50 includes a power supply, lines that connect the power supply to the terminals 11a, 12a, and 13a, and a switch provided in the lines. For example, one power supply is electrically connected to the first electrode 11 and the second electrode 12 through the lines and terminals 11a and 12a. The voltage applicator 50 changes the voltage applied to the first electrode 11 and the second electrode 12 by inverting the polarity of the power supplies connected to the first electrode 11 and the second electrode 12 using a switch. For example, each of the two power supplies may be electrically connected to both the first electrode 11 and the second electrode 12 through lines and terminals 11a and 12a. The voltage applicator 50 may change the voltage applied to the first electrode 11 and the second electrode 12 by switching the power supplies to which the first electrode 11 and the second electrode are connected using a switch.

The controller 70 performs information processing to control the voltage application with the voltage applicator 50. The controller 70 includes, for example, a processor, a microcomputer, or a dedicated circuit. When the enzymatic reaction device 100 includes a stirring mechanism, the controller 70 may control the stirring mechanism.

### Operation

The operation of the enzymatic reaction device 100 according to the present embodiment, in other words, a method for performing an enzymatic reaction using the enzymatic reaction device 100 will be described below.

In the enzymatic reaction device 100, the voltage applicator 50 applies a voltage to the first electrode 11 and the second electrode 12, thereby causing the reaction of the target molecule contained in the sample contained in the first container 21. FIG. 8 illustrates an example of a voltage application pattern to the first electrode 11 and the second electrode 12 in the enzymatic reaction device 100 according to the present embodiment. The upper side of FIG. 8 illustrates a voltage application pattern to the first electrode 11 with the voltage applicator 50, and the lower side of FIG. 8 illustrates a voltage application pattern to the second electrode 12 with the voltage applicator 50. In FIG. 8, the vertical axis represents the absolute value of the voltage applied to the first electrode 11 or the second electrode 12 with respect to the potential of the reference electrode 13. In FIG. 8, the horizontal axis represents time, and the reaction start time point is set to 0.

As illustrated in FIG. 8, the voltage applicator 50 applies a voltage to the first electrode 11 and the second electrode 12 in such a manner that a first voltage application period T1 and a second voltage application period T2, in which the magnitudes of the voltages applied to the first electrode 11 and the second electrode 12 are different from each other, are alternately repeated. For example, the controller 70 controls the switch included in the voltage applicator 50 to change the voltage applied to the first electrode 11 and the second electrode 12. When the first voltage application period T1 ends, the voltages applied to the first electrode 11 and the second electrode 12 are changed, and the second voltage application period T2 starts. When the second voltage application period T2 ends, the voltages applied to the first electrode 11 and the second electrode 12 are changed, and the first voltage application period T1 starts. In the example illustrated in FIG. 8, the voltage application with the voltage applicator 50 starts from the first voltage application period T1. However, the voltage application may start from the second voltage application period T2.

In the first voltage application period T1, the voltage applicator 50 applies a voltage to the first electrode 11 in such a manner that the first electrode 11 functions as a working electrode that causes the reaction of the target molecule. When the enzyme 36 reduces the target molecule, the voltage applicator 50 applies, for example, a voltage of −2 V or a negative voltage higher than-2 V to the first electrode 11. This donates electrons from the electrode body 30 to the enzyme 36, thereby regenerating the reducing power of the enzyme 36 that was lost by reducing the target molecule. When the enzyme 36 oxidizes the target molecule, the voltage applicator 50 applies, for example, a voltage of 2 V or a positive voltage lower than 2 V to the first electrode 11. This regenerates the oxidizing power of the enzyme 36 that was lost by oxidizing the target molecule because the electrode body 30 accepts electrons from the enzyme 36. In this case, the voltage applicator 50 applies a voltage, which does not cause the second electrode 12 to function as a working electrode, to the second electrode 12, for example, a voltage that causes the second electrode 12 to function as a counter electrode to the working electrode. Specifically, the voltage applicator 50 applies a voltage having an absolute value smaller than that of the voltage applied to the first electrode 11, to the second electrode 12, such as 0 V or a voltage close to 0 V, for example, 0 V±0.1 V.

In the second voltage application period T2, the voltages applied to the first electrode 11 and the second electrode 12 are reversed from those in the first voltage application period T1. Specifically, the voltage applicator 50 applies a voltage to the second electrode 12 in such a manner that the second electrode 12 functions as a working electrode that causes a reaction of the target molecule. When the target molecule is reduced, the voltage applicator 50 applies, for example, a voltage of −2 V or a negative voltage higher than −2 V to the second electrode 12. When the target molecule is oxidized, the voltage applicator 50 applies, for example, a voltage of 2 V or a positive voltage lower than 2 V to the second electrode 12. For example, the voltage applicator 50 applies a voltage having the same magnitude as the voltage applied to the first electrode 11 in the first voltage application period T1, to the second electrode 12 in the second voltage application period T2. In this case, the voltage applicator 50 applies a voltage that does not cause the first electrode 11 to function as a working electrode, to the first electrode 11, for example, a voltage that causes the first electrode 11 to function as a counter electrode to the working electrode. Specifically, the voltage applicator 50 applies a voltage having an absolute value smaller than that of the voltage applied to the second electrode 12, to the first electrode 11, such as 0 V or close to 0 V, for example, 0 V±0.1 V.

As described above, the method for performing an enzymatic reaction using the first electrode 11 and the second electrode 12 includes applying a voltage to the first electrode 11 so that the first electrode 11 functions as a working electrode that causes the reaction of the target molecule (first voltage application period T1), and applying a voltage to the second electrode 12 so that the second electrode 12 functions as a working electrode that causes the reaction of the target molecule (second voltage application period T2), in which the applying the voltage to the first electrode and the applying the voltage to the second electrode are repeated.

During the first voltage application period T1, the enzyme 36 causes the reaction of the target molecule on the first electrode 11. That is, the first electrode 11 functions as a working electrode. The enzyme 36 is repeatedly activated by the voltage applied to the first electrode, so that the enzymatic reaction proceeds efficiently. Due to the effect of the voltage applied to the first electrode 11, molecules other than the target molecule in the sample are electrically attracted to and adsorbed onto the first electrode 11.

During the second voltage application period T2, the enzyme 36 causes the reaction of the target molecule on the second electrode 12. The enzyme 36 is repeatedly activated by the voltage applied to the second electrode, so that the enzymatic reaction proceeds efficiently. That is, the second electrode 12 functions as a working electrode. Due to the effect of the voltage applied to the second electrode 12, molecules other than the target molecule in the sample are electrically attracted to and adsorbed onto the second electrode 12.

The repetition of the first voltage application period T1 and the second voltage application period T2 inhibits the adsorption of molecules other than the target molecule in the sample onto the second electrode 12 during the first voltage application period T1, and inhibits the adsorption of molecules other than the target molecule in the sample onto the first electrode 11 during the second voltage application period T2. This can inhibit a decrease in the efficiency of the enzymatic reaction caused by the adsorption of such molecules over time. Furthermore, because one of the first electrode 11 and the second electrode 12 functions as a working electrode during the first voltage application period T1 and the second voltage application period T2, sufficient time is ensured for the enzymatic reaction to occur, enabling the target molecule in the sample to react with high efficiency.

Even when protons are consumed or generated by the enzymatic reaction, one of the first electrode 11 and the second electrode 12 functions as a working electrode, and the other functions as a counter electrode. Thus, protons are generated or consumed at the counter electrode in the opposite manner to that at the working electrode, thereby inhibiting a change in pH during the operation of the enzymatic reaction device 100. In particular, in each of the first and second electrodes 11 and 12, the period of functioning as the working electrode and the period of functioning as the counter electrode are alternately repeated. Thus, changes in local pH around the first and second electrodes 11 and 12 can also be reduced. For example, when the sample is food, a reduction in the changes in pH can inhibit unintended food alterations.

As illustrated in FIG. 8, for example, the length of the first voltage application period T1 and the length of the second voltage application period T2 are the same. This allows the reaction of the target molecules to occur evenly at the first electrode 11 and the second electrode 12, making it possible to more efficiently cause the reaction of the target molecules. In this specification, the phrase “the length of the first voltage application period T1 and the length of the second voltage application period T2 are the same” indicates that they are substantially the same. For example, the difference between the length of the first voltage application period T1 and the length of the second voltage application period T2 is less than or equal to 3% with respect to each of the length of the first voltage application period T1 and the length of the second voltage application period T2.

The length of each of the first voltage application period T1 and the second voltage application period T2 is, for example, greater than or equal to 0.5 minutes and less than or equal to 30 minutes. This makes it possible to effectively inhibit the adsorption of molecules other than the target molecule to the first electrode 11 and the second electrode 12 while ensuring the time necessary for stabilization of the reaction of the target molecule at the first electrode 11 and the second electrode 12. The length of each of the first voltage application period T1 and the second voltage application period T2 may be greater than or equal to 1 minute and less than or equal to 15 minutes.

Between the first voltage application period T1 and the second voltage application period T2, there may be a period other than the first voltage application period T1 and the second voltage application period T2. For example, between the first voltage application period T1 and the second voltage application period T2, there may be a period during which no voltage is applied to the first electrode 11 or the second electrode 12 with the voltage applicator 50. The length of the period other than the first voltage application period T1 and the second voltage application period T2 is, for example, less than or equal to 10% of the total length of the first voltage application period T1 and the second voltage application period T2.

### Variations

An enzymatic reaction device according to a variation of the present embodiment will be described below. Hereinafter, differences from the embodiment will be mainly described, and the descriptions of configurations in common will be omitted or simplified.

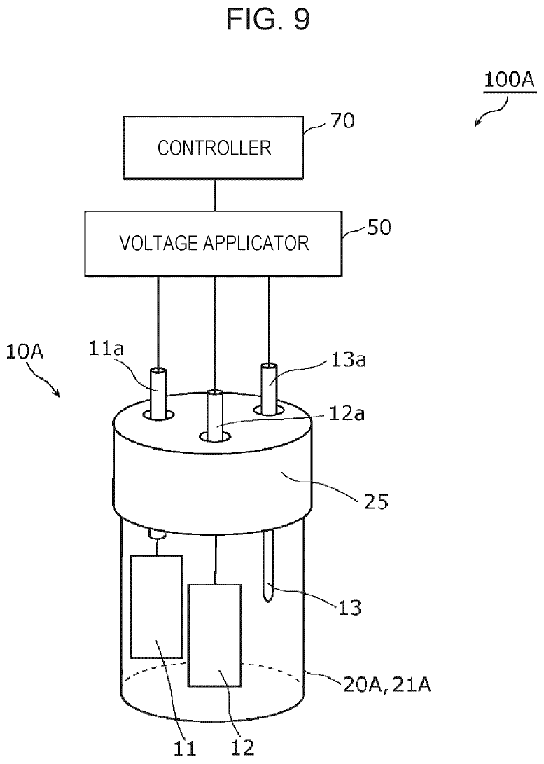

FIG. 9 illustrates the configuration of an enzymatic reaction device according to the present variation. In FIG. 9, the first electrode 11, the second electrode 12, and the reference electrode 13 arranged inside a reaction vessel 20A are indicated by solid lines, and portions of the reaction vessel 20A that cannot be seen from the surface are indicated by dashed lines.

As illustrated in FIG. 9, an enzymatic reaction device 100A according to the variation is different from the enzymatic reaction device 100 according to the embodiment in that a reactor 10A is provided instead of the reactor 10. The reactor 10A has a configuration in which the reaction vessel 20A is provided instead of the reaction vessel 20 of the reactor 10.

The reaction vessel 20A includes one container 21A. The first electrode 11, the second electrode 12, and the reference electrode 13 are disposed inside the container 21A. The container 21A contains a sample containing a target molecule. In this manner, the first electrode 11, the second electrode 12, and the reference electrode 13 may be placed in one container 21A to perform an enzymatic reaction.

In the enzymatic reaction device 100A, as in the enzymatic reaction device 100, the voltage applicator 50 applies a voltage to the first electrode 11 and the second electrode 12 in such a manner that the above-described first voltage application period T1 and second voltage application period T2 are alternately repeated. Therefore, the enzymatic reaction device 100A can also cause the target molecule in the sample to react with high efficiency.

### Example

An enzymatic reaction device and a method for performing an enzymatic reaction according to an embodiment of the present disclosure will be specifically described below in the following example. However, the following example is merely an example, and the present disclosure is not limited to the following example.

### Enzymatic Reaction

An enzymatic reaction in each of the example and a comparative example was performed by the following method.

### (1) Example

In the enzymatic reaction in the example, first, the reactor 10 and a potentiostat illustrated in FIGS. 5 to 7 were prepared, the potentiostat being included in the voltage applicator 50. The working electrode terminal of the potentiostat was connected to the first and second electrodes 11 and 12 with a switch that controlled the connection and disconnection between the working electrode terminal and the first and second electrodes 11 and 12. The counter electrode terminal of the potentiostat was connected to the first and second electrodes 11 and 12 with a switch that controlled the connection and disconnection between the counter electrode terminal and the first and second electrodes 11 and 12. The reference electrode terminal of the potentiostat was connected to the reference electrode 13.

As each of the first electrode 11 and the second electrode 12, an enzyme-immobilized electrode containing ferredoxin-thioredoxin reductase serving as the enzyme 36 and methyl viologen serving as the electron carrier 37 was used. The enzyme 36 and the electron carrier 37 were immobilized on the conductive layer 33 made of gold using the first linker 41 containing an alkyl chain having 4 carbon atoms and the second linker 42 containing an alkyl chain having 10 carbon atoms. As the reference electrode 13, a silver/silver chloride electrode was used.

Milk was contained as a sample in the first container 21. Phosphate-buffered saline with a pH of 7.4 was contained as a standard solution in the second container 22. A voltage was applied to the first electrode 11 and the second electrode 12 according to the voltage application pattern illustrated in FIG. 8, and the enzymatic reaction was performed for 8 hours. In this case, the length of the first voltage application period T1 and the second voltage application period T2 was 1 minute each. When the first electrode 11 or the second electrode 12 was used as a working electrode, the potential of the first electrode 11 or the second electrode 12 relative to the reference electrode 13 was controlled by the potentiostat so as to be equal to the electron carrier 37.

### (2) Comparative Example

In the enzymatic reaction of the comparative example, the reactor 110 and the potentiostat illustrated in FIG. 1 were prepared. The working electrode terminal of the potentiostat was connected to the working electrode 111. The counter electrode terminal of the potentiostat was connected to the counter electrode 112. The reference electrode terminal of the potentiostat was connected to the reference electrode 113. The same enzyme-immobilized electrode as each of the first and second electrodes 11 and 12 in the example was used as the working electrode 111. A platinum electrode was used as the counter electrode. A silver/silver chloride electrode was used for the reference electrode 113.

Milk was contained as a sample in the first container 121. Phosphate-buffered saline with a pH of 7.4 was contained as a standard solution in the second container 122. A voltage was applied to the working electrode 111 according to the voltage application pattern illustrated in FIG. 2, 0 V was applied to the counter electrode 112, and the enzymatic reaction was performed for 8 hours. That is, a constant voltage was continuously applied to the working electrode 111 during the enzymatic reaction. The voltage applied to the working electrode 111 was controlled by the potentiostat so that the potential of the working electrode 111 relative to the reference electrode 113 was equal to the reduction potential of the electron carrier 37.

### Determination of Reduction Rate of Disulfide Bond in Target Molecule

The target molecule was β-lactoglobulin in milk. The reduction rate of the disulfide bonds in β-lactoglobulin was determined. The disulfide bonds in β-lactoglobulin are cleaved by reduction.

Specifically, samples taken 4 and 8 hours after the start of the reaction were reacted with a digestive enzyme at 37° C. for 30 minutes, and then subjected to sodium dodecyl sulfate-polyacrylamide gel electrophoresis (SDS-PAGE). The gel after electrophoresis was then stained. The area and intensity of the β-lactoglobulin band were quantified by image analysis. SDS-PAGE and image analysis were also performed on untreated milk that had not been subjected to the enzymatic reaction. The resulting value was defined as the value corresponding to a 100% residual rate of the target molecule. The residual rate of β-lactoglobulin after the enzymatic reaction was calculated by proportional calculation. The reduction rate of β-lactoglobulin after the enzymatic reaction was calculated as “100%-residual rate=reduction rate”.

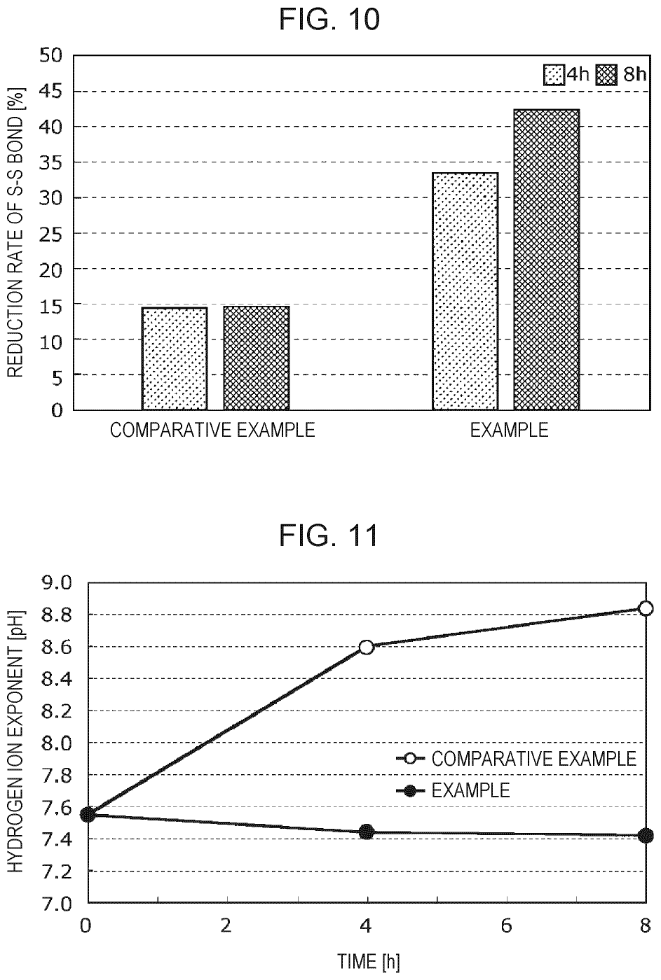

FIG. 10 is a graph illustrating the reduction rates of disulfide bonds (S-S) of β-lactoglobulin in the example and the comparative example. As illustrated in FIG. 10, in the comparative example, the reduction rate remains almost unchanged between 4 hours and 8 hours after the start of the reaction, and the reduction rate reaches a ceiling at about 15%. This is presumably due to the adsorption of molecules other than β-lactoglobulin on the working electrode 111, as described above. In contrast, in the example, the reduction rate at both 4 hours and 8 hours after the start of the reaction is higher than that in the comparative example. Furthermore, the enzymatic reaction continues beyond 4 hours after the start of the reaction, resulting in an increase in the reduction rate. That is, in the example, the target molecules in the sample can be reacted with high efficiency. This is presumably because the repetition of the first voltage application period T1 and the second voltage application period T2 allows sufficient time for the enzymatic reaction to occur while molecules other than β-lactoglobulin are less likely to adsorb onto the first and second electrodes 11 and 12.

### Change in pH

The pH of the samples was measured using a pH meter at the start of the reaction and 4 and 8 hours after the start of the reaction. FIG. 11 illustrates changes in the pH of samples in the example and the comparative example. pH may refer to the hydrogen ion exponent. In FIG. 11, the vertical axis represents the pH of the sample, and the horizontal axis represents the time elapsed from the start of the reaction. As illustrated in FIG. 11, in the comparative example, the pH increases with time, whereas in the example, the pH remains almost unchanged even over time. This is presumably because, in both the first voltage application period T1 and the second voltage application period T2, one of the first electrode 11 and the second electrode 12 functions as a working electrode, and the other functions as a counter electrode.

### OTHER EMBODIMENTS

The enzymatic reaction device and the method for performing an enzymatic reaction according to an embodiment of the present disclosure have been described above based on the embodiments and the example, but the present disclosure is not limited to these embodiments and the example. Various modifications conceived by those skilled in the art and applied to the embodiments and examples, as well as alternative embodiments constructed by combining some of the components described in the embodiments, are included within the scope of the present disclosure, as long as they do not depart from the gist of the present disclosure.

For example, in the above embodiment, the second electrode 12 functions as a counter electrode in the first voltage application period T1, and the first electrode 11 functions as a counter electrode in the second voltage application period T2. However, the present disclosure is not limited thereto. For example, another electrode that functions as a counter electrode other than the first electrode 11 and the second electrode 12 may be provided in the reactor. In this case, for example, during the first voltage application period T1, the voltage applicator 50 applies a voltage to the first electrode 11 in such a manner that the first electrode 11 functions as a working electrode, and applies a voltage to the other electrode in such a manner that the other electrode functions as a counter electrode. For example, during the second voltage application period T2, the voltage applicator 50 applies a voltage to the second electrode 12 in such a manner that the second electrode 12 functions as a working electrode, and applies a voltage to the other electrode in such a manner that the other electrode functions as a counter electrode.

The enzymatic reaction device and the method for performing an enzymatic reaction according to an embodiment of the present disclosure are useful for converting a target molecule in a sample. For example, the enzymatic reaction device and the method for performing an enzymatic reaction according to an embodiment of the present disclosure can be used for various applications, such as selective conversion of some components in food.

What is claimed is:

1. An enzymatic reaction device, comprising:
a first electrode and a second electrode each including:
at least one of an enzyme or a coenzyme that causes a reaction of a target molecule in a sample, and
an electrode body including at least one of the enzyme or the coenzyme immobilized on a surface of the electrode body; and
a voltage applicator that applies voltages to the first electrode and the second electrode,
wherein the voltage applicator applies a voltage to the first electrode and the second electrode in such a manner that a first voltage application period during which a voltage is applied to the first electrode so that the first electrode functions as a working electrode that causes a reaction of the target molecule, and a second voltage application period during which a voltage is applied to the second electrode so that the second electrode functions as a working electrode that causes a reaction of the target molecule, are alternately repeated.

2. The enzymatic reaction device according to claim 1, wherein an oxidoreductase that oxidizes or reduces the target molecule is immobilized on the surface of the electrode body as the enzyme.

3. The enzymatic reaction device according to claim 2, wherein each of the first electrode and the second electrode includes:
an electron carrier that transports an electron between the electrode body and the oxidoreductase,
a first linker in a form of a chain, and
a second linker in a form of a chain, the second linker being longer than the first linker,
wherein the electron carrier is immobilized on the surface of the electrode body with the first linker interposed therebetween, and
the oxidoreductase is immobilized on the surface of the electrode body with the second linker interposed therebetween.

4. The enzymatic reaction device according to claim 1, wherein a length of the first voltage application period is identical to a length of the second voltage application period.

5. A method for performing an enzymatic reaction using a first electrode and a second electrode each including at least one of an enzyme or a coenzyme that causes a reaction of a target molecule in a sample and an electrode body including at least one of the enzyme or the coenzyme immobilized on a surface of the electrode body, the method comprising:
applying a voltage to a first electrode so that the first electrode functions as a working electrode that causes a reaction of a target molecule; and
applying a voltage to a second electrode so that the second electrode functions as a working electrode that causes a reaction of the target molecule,
wherein the applying the voltage to the first electrode and the applying the voltage to the second electrode are alternately repeated.

6. The method for performing an enzymatic reaction according to claim 5, wherein the sample is food.
